(19) United States
(12) Patent Application Publication
(10) Pub. No.: US 2007/0198041 A1
(43) Pub. Date: Aug. 23, 2007

(54) Tissue Or Organ Manipulation Device

(76) Inventors: Heinz Rupp, Marburg (DE); Bernhard Maisch, Marburg (DE); Ekkehard Schuler, Marburg (DE); Michael Koch, Steffenberg (DE)

Correspondence Address: CLARK & BRODY, 1090 VERMONT AVENUE, NW, SUITE 250, WASHINGTON, DC, 20005, US

(21) Appl. No.: 10/568,430

(22) PCT Filed: Aug. 12, 2004
(86) PCT No.: PCT/DE04/01806

§ 371 Date: Oct. 20, 2006

(30) Foreign Application Priority Data
Aug. 14, 2003 (DE) 10337813.8

(51) Int. Cl. A61B 17/32

(52) U.S. Cl. 606/184

(57) ABSTRACT

The invention relates to apparatus puncturing, or manipulating in some other way, human or animal tissues, tissue attachment being reliably detected and communicated. In particular the apparatus of the invention allows accessing the cardiac sac after the pericardium has been pierced. The apparatus of the invention is applicable to any human or animal or organ tissue, foremost within the scope of minimally invasive surgery lacking direct visual monitoring and where it is necessary to make sure that tissue/organs be attached for purposes of manipulation, in particular puncturing, to a special, for instance puncturing apparatus. The invention is not restricted to living tissue/organs.

The present invention relates to apparatus puncturing or manipulating in some other way human or animal tissue, tissue attachment being reliably detected and communicated. In particular the apparatus of the invention allows accessing the pericardial cavity after piercing the pericardium.

U.S. Pat. No. 5,972,013 describes a device allowing minimally invasive access to the human or animal heart pericardium. This device comprises a penetrating element configured within a lumen of a guide tube. This guide tube is fitted at its distal end with a deflecting mechanism to deflect the penetrating element's distal end. Moreover said guide tube is fitted at its distal end with a head having a lateral aperture to receive the tissue to be punctured. For that purpose a partial vacuum source is connected to the guide tube, which furthermore may be made of plastic to attain flexibility while still allowing applying a partial vacuum to the pericardium only when the said head or said aperture is properly positioned relative to it. Once the treating physician is assured that the pericardium has been attached to the aperture, the penetrating element is deflected through the deflecting mechanism, that is, it will be made to pierce at a slant the attached pericardium.

U.S. Pat. No. 5,931,810 A also describes a device allowing entering the pericardium. This device consists of an element fitted with both a distal and a proximal end. Said element comprises a continuous borehole in it. The proximal end is fitted with several clamping jaws that can be opened and closed. At least one of said clamping jaws is displaceable. The distal end is fitted with a grip which is partly linked to the displaceable jaw, whereby the jaw can be opened/closed at any desired time. A tissue-puncturing needle is housed within the jaw and can be displaced within said continuous borehole. Moreover said needle is linked to a stop limiting its is displacements.

The two above devices incur the drawback that attaching the body or organ tissue to said device(s) is neither reliably detected nor communicated. However attachment detection is the precondition for successful puncturing or any other manipulation. Moreover said above devices do not provide reliable detection whether the tissue or organ to be punctured was attached to the proper head position, for instance to the side aperture. There is danger therefore of injuring the critical organs or tissues, for instance the cardiac muscle, during manipulation.

The objective of the present invention is to create apparatus meeting the preconditions of successful puncture or other manipulation of human or animal tissue.

Said objective is attained in the present invention by apparatus applicable to human medicine or veterinary art as defined in claim 1.

Therefore apparatus has been developed to solve the above problems in reliably detecting and communicating adequate attachment of tissue or organs to carry out successful puncturing or other manipulations.

The apparatus of the present invention may be used for any human or animal tissue or organ foremost within the scope of minimally invasive surgery where visible checks are absent and where assurance is needed that a tissue or organ is attached to a specific structure, for instance a puncturing apparatus, for purposes of manipulation.

The developed apparatus of the invention consists of a partial vacuum source, of a suction head fitted with a recess, at least one penetrating element housed in at least one guide device having at least one lumen, and both an attachment detection system and a display unit.

The attachment detection system of the present invention may comprise one or more detectors among which acoustic, optical or pressurized detectors. Signals sensed by the attachment detection system can be converted into display signals by an appropriate display unit.

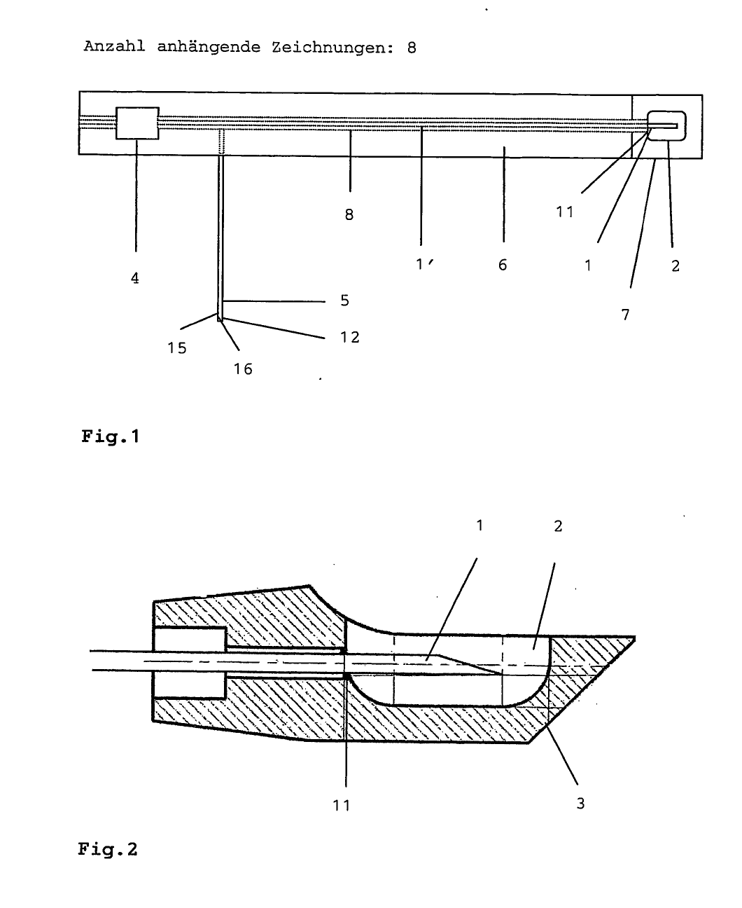

Illustrative embodiments of the present invention are shown in FIGS. 1 through 8.

FIG. 1 shows all components of the apparatus of the invention.

FIG. 2 is a sideview of the suction head 7,

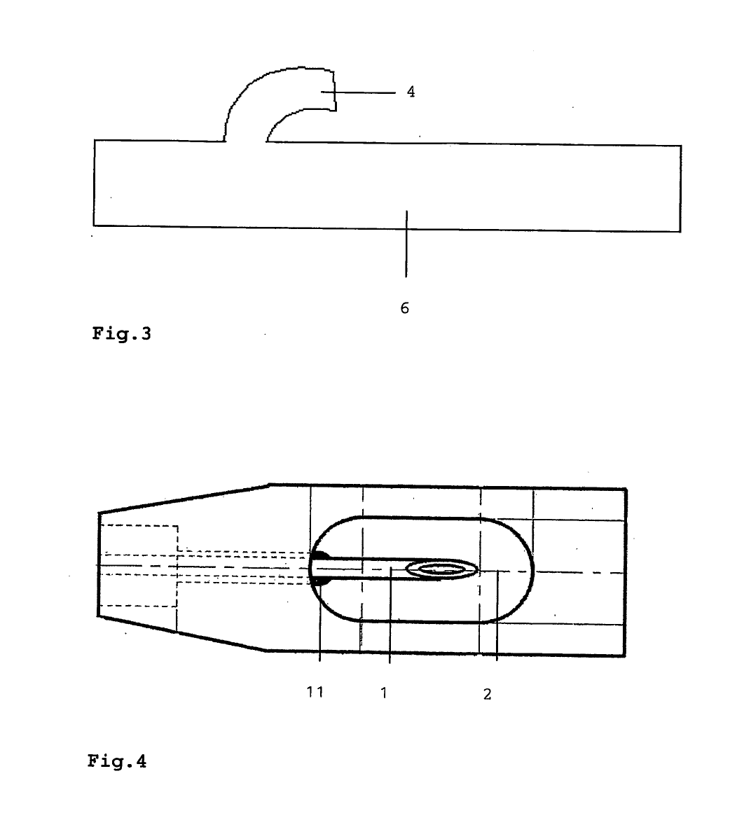

FIG. 3 shows the control unit 4,

FIG. 4 shows the suction head 7 of FIG. 2,

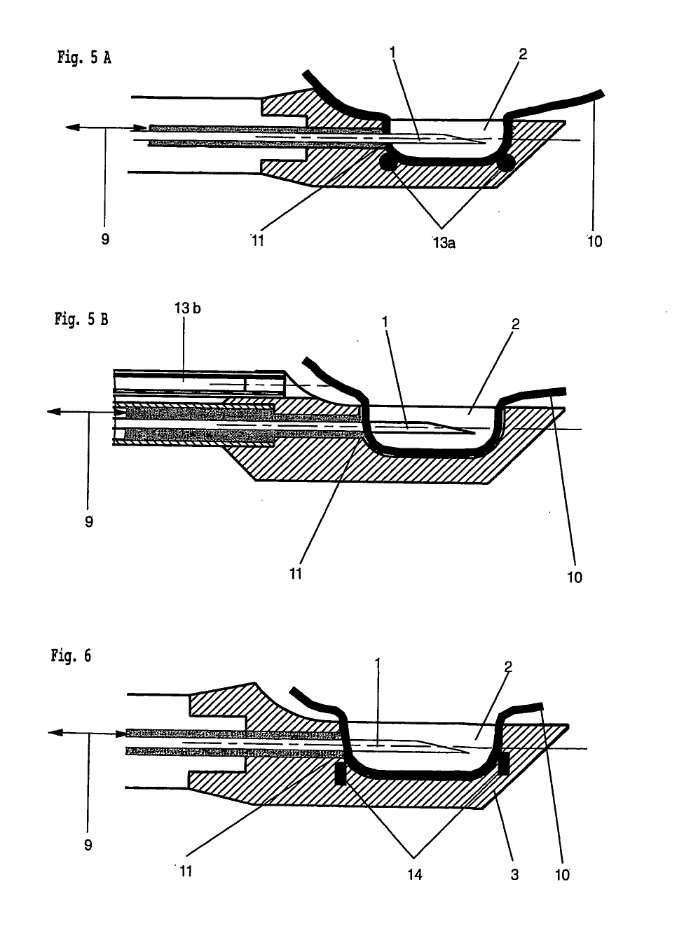

FIGS. 5a, 5b show the tissue or organ 10 aspirated by the apparatus of the invention, the attachment detection system 12 comprising optical penetration detectors for instance in the form of an interruptible light beam 13a and/or an endoscope 13b,

FIG. 6 shows the suction head 7 comprising a detector in the form of an ultrasonic transmitter and receiver unit, i.e. a transceiver 14,

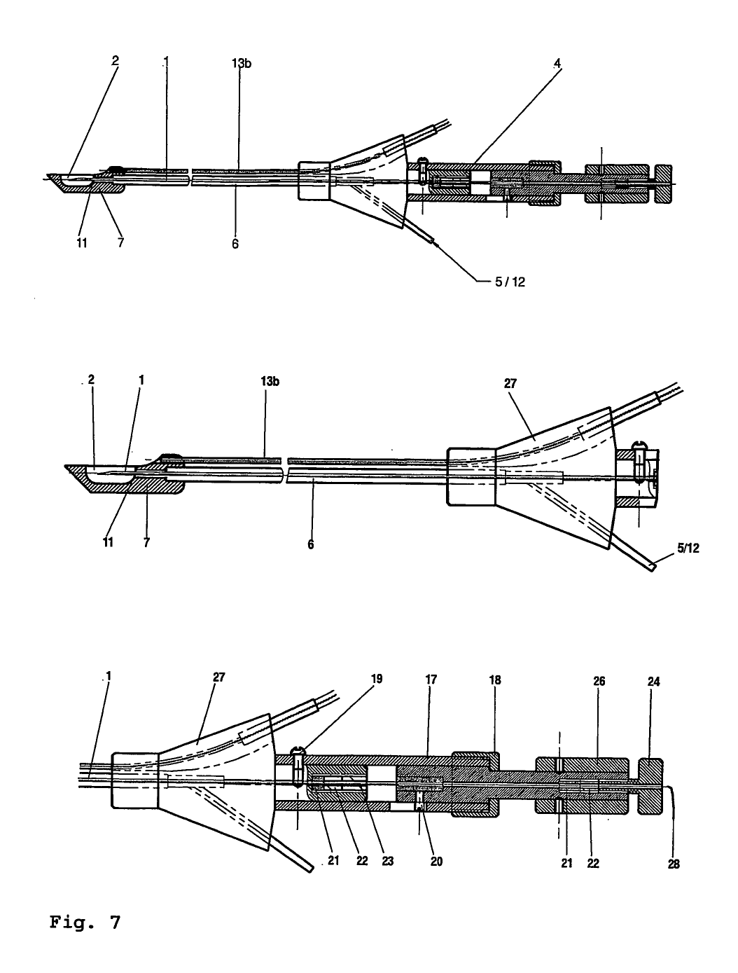

FIG. 7 shows a complete embodiment mode with a rigid guide device, ready for use on a patient, and

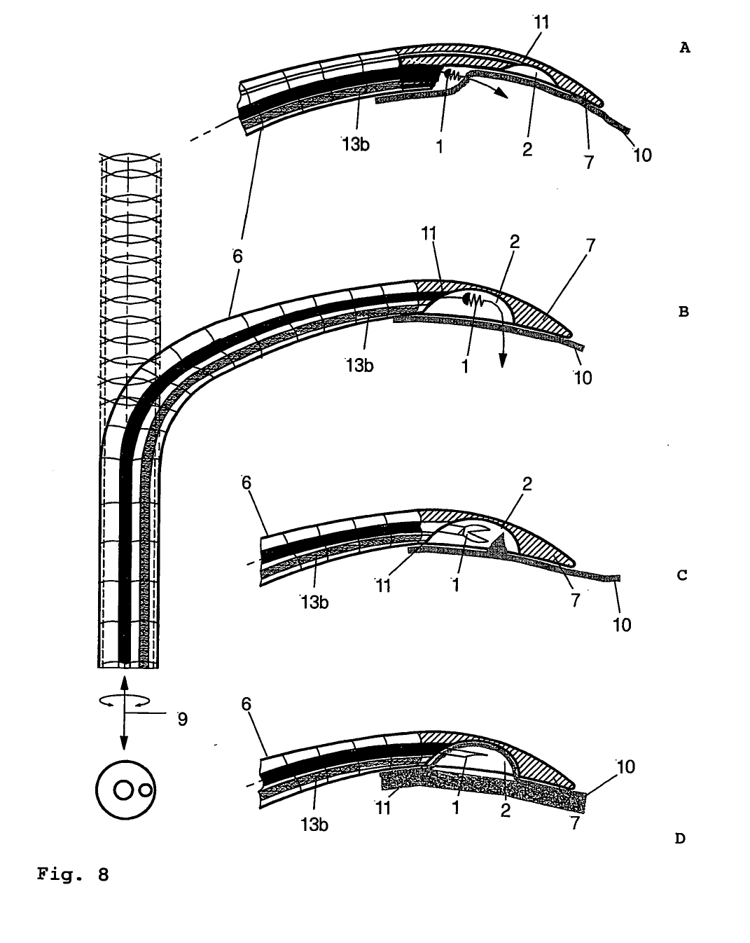

FIG. 8 shows the (exchangeable) head of an illustrative embodiment comprising a flexible guide device.

These Figures are elucidated below.

FIG. 1 shows all the components of the apparatus of the invention. The guide device 6 fitted with the penetrating element 1, for instance a needle, scissors, tongs or electrode or another manipulating item, the control unit 4 for the penetrating element 1, terminates proximally in a suction head 7. The partial vacuum source 5, the attachment detection system 12 and the display unit 16 are connected to the apparatus of the invention. In this preferred embodiment mode, the attachment detection system 12 is fitted with a pressure sensor 15 to detect attachment

The guide device 6 comprises a distal and a proximal end. The suction head 7 is situated at said element's proximal end. Furthermore the suction head 7 is fitted with a recess 2 having a lateral aperture that aspirates the tissue or organ to be punctured. The pressure detector may be configured within the suction head 7 inside the partial vacuum source 5 or at any site in the partial vacuum duct between the suction head 7 and said source 5.

To enable puncturing or other manipulations such as irradiation, the penetrating element 1 may be advanced/retracted along its longitudinal axis, or be rotated about it, inside the guide device 6, by a control unit 4. The penetrating element 1 is inserted into the apparatus of the invention at its distal end. A stop is provided to keep the penetrating element in position “before puncture”. Once tissue or organ attachment has been reliably detected, the penetrating element 1 can be inserted into said tissue/organ. The stop may be a simple mechanical means, for instance a clamp.

Preferably the partial vacuum source 5 is a continuously operating suction pump (without compensation for any changes in the ambient pressure) or a continuously operating source of partial vacuum in order to compensate any leakages during attachment. Attachment detection is attained by prior calibration or adjustment by measuring the pressure when the suction head is closed.

A pressure-dependent detector such as a pressure sensor 15 measures the pressure changes. It is preferably situated outside the apparatus of the invention where the measurement line takes up a minimum of dead space. The pressure sensor 15 offers high resolution in time in order to record pressure changes. As long as attachment has not taken place, the suction of the partial vacuum source is unimpeded. A rise in negative pressure indicates that the tissue/organ 10 to be punctured rests on the vacuum aperture 11 in the suction head 7, i.e., the precondition of successful manipulation has been met. Such precondition can be detected by an attachment detection system 12 employing various means.

All signals detected by the detection system such as pressure, light (FIG. 5) etc. are converted into display signals by a display unit 15. These displays signals may be optical, acoustic, olfactory or taste or tactile.

FIG. 2 is a lateral section of the suction head 7 fitted with a penetrating element such as a needle 1 that is displaced forward/backward along its longitudinal axis and rotated about latter and stopped by a control unit 4. The recess subtending the lateral aperture 2 in the suction head 7 is elongated in this embodiment mode. The proximal end of the suction head 7 is oblique.

The partial vacuum aperture 11 in the suction head 7 entirely encloses the puncturing needle. The partial vacuum duct within the guide device 6 preferably tapers on the way toward the partial vacuum aperture 11 in the suction head 7. Other partial vacuum apertures besides the aperture 11 may be present in the suction head.

FIG. 3 illustratively shows the control unit 4 in the form of an integral arcuate structure allowing to move forward/back the penetrating element 1 and also to rotate it or stopping it. The stop of this embodiment mode is a simple clamp. For that purpose the clamp 4 is moved into a tapering slot of the guide device 6 until it jams in place.

FIG. 4 is a topview of the suction head 7 being fitted with a penetrating element such as a needle 1 of FIG. 2.

FIG. 5 shows the tissue/organ 10 which is aspirated by partial vacuum into the recess 2 of the suction head 7, the attachment detection system 12 comprising an optical detector, for instance an interruptible light beam 13a. In this embodiment mode the tissue 10 is aspirated by a partial vacuum source 5 into the recess 2 and attachment is detected by an interruptible light beam 13a. As soon as the tissue/organ enters the recess and thus interrupts the light source signal, this event is detected by the interruptible light beam and then is displayed by the display unit 16.

FIG. 6 shows the suction head 7, attachment of the tissue/organ 10 aspirated into the recess 2 being detected by an ultrasonic sensor fitted with a reflector or with an ultrasonic transceiver 14 fitted with the detector in the form of the attachment detection system 12. The ultrasonic beam is emitted by an ultrasonic transmitter and received by an ultrasonic receiver. As soon as the tissue/organ moves between said transmitter and receiver, the received signal intensity will change.

FIG. 7 shows a further embodiment mode comprising a rigid guide device 6. In this embodiment mode, said guide device comprises—besides the lumen guiding the penetrating element 1 and applying the partial pressure to the suction head 7—a further borehole or a further lumen to receive an endoscope (13b) within the attachment detection system. This further borehole is shown at the top of the upper partial view. The lumen guiding the penetrating element and applying the partial pressure between the suction head and the additional pressure-dependent (omitted) detector of this particular embodiment mode is situated underneath, its exit being denoted by “5/12”. This endoscope also may be advantageously used to ascertain the optimal attachment site.

The center drawing shows a junction 27 linking the guide device 6 to the control unit 4 of the penetrating element 1. The junction 27 is designed to maintain and transmit a partial vacuum between the suction head and the partial vacuum source and preferably can be repeatedly removed from the guide device and, in an omitted, particular and especially preferred embodiment mode, it comprises adapters to mount partial vacuum hoses (at the bottom) and fiber optics for optical detectors in the form of an interruptible light beam or an endoscope that in conventional manner is mostly made of light guiding fibers.

The lower partial drawing shows an embodiment mode for a control unit 4. In this embodiment mode the penetrating element 1 is connected to a guide shaft 25 or a guide wire 28, said control unit otherwise comprising a guide device 17 for rotations and diverse stop and guide screws 18, 19, 20, 23, 24 around illustratively silicone seals 21 and a compression roller in a manner that the penetrating element 1 can be moved forward/back approximately parallel to the guide device and also be rotated. In the event that the attachment detection system is in the form of pressure and pressure change detectors, the control unit 4 shall be so matched to said detectors that, to preclude “false positive” attachment, the displacement of the penetrating element shall entail only minimal pressure changes.

FIG. 8 shows the (exchangeable) head of an embodiment mode having a flexible guide device while using a controlled endoscope, a pressure-sensitive detector and an optic detector (in this instance an endoscope 13b) being used. Illustrative embodiments of the penetrating element are a screw electrode (FIG. 8b), scissors (FIG. 8c) and a needle (FIG. 8d). In embodiment modes a-c, a rigid tissue 10 is attached in controlled manner only to the head. In embodiment d, an elastic tissue is attached into the recess, the penetrating element being a needle for instance to access the cardiac sac.

FIG. 8a shows another embodiment mode wherein—besides the lumen applying a partial vacuum between the source and the suction head and the lumen guiding the optical detectors in the form of an endoscope 13b—the guide device contains a further lumen guiding a further penetrating element, for instance a screw electrode. In this manner electrodes of a defibrillator or of a cardiac pacemaker may be positioned in the vicinity of the heart or, in targeted manner, on the heart.

The third lumen guiding a further penetrating element obviously may be configured within a second guide device which is detachably connected to the first guide device.

The optic detectors may be fiber optics of which the optical materials are electrically conducting so that electrical monitoring and identification of the tissue to be attached can be carried out in parallel with optical monitoring. The simplest approach consists in affixing electrodes slightly penetrating the tissue. By applying a voltage and by subsequently measuring the electrical impedance between electrodes in the form of slightly penetrating elements, the attached tissue can be easily identified. Obviously very simple non-penetrating electrodes may also be used as detectors. Both penetrating and non-penetrating electrodes allow tissue identification by means of electrical or combined electrical and optical measurements, the tissue being recognized in the case of electrical measurements for instance on account of impedance differentials. Also electrical detectors or penetrating electrodes allow monitoring organ functions to the extent they transmit or require electrical signals.

A further, omitted embodiment mode provides that the display unit comprise an optic display element, for instance LED's, by means of which the signal from the attachment detection system 12 can be converted into an optical signal.

A further, omitted embodiment mode provides that a sound source is used as the display unit. In this case the signal from the attachment detection system 12 is converted into an acoustic signal, a higher voltage for instance corresponding to a louder acoustic signal. This feature offers the advantage that the observer will not be distracted by monitoring the pressure sensor acting as the detector where one would be used for such purposes. Similar considerations apply when a display unit is used in the form of a vibrator (tactile signal).

### LIST OF REFERENCE NUMERALS

1 penetrating element (for instance needle)
1′ needle (not visible)
2 recess
3 oblique end of suction head
4 control unit for penetrating element
5 partial vacuum source
6 guide device
7 suction head
8 lumen
9 needle displacement direction
10 aspirated tissue
11 vacuum aperture
12 attachment detection system
13a light barrier
13b endoscope
14 ultrasonic sensor
15 pressure sensor
16 display unit
17 rotation guide element
18 screw affixation
19 rotation stop screw
20 forward displacement guide screw
21 silicone seal
22 compression roller
23 compression screw
24 compression screw
25 penetrating element guide shaft
26 grip
27 junction
28 guide wire.

1. Apparatus for puncturing or otherwise manipulating human or animal tissue or organs, comprising at least one guide device (6) for instance in the form of a rigid or flexible tube, at least one penetrating element (1) which is guided within the guide device (6) and which is displaceable forward and backward and rotatable within this guide device (6) by a control unit (4), further a partial vacuum source (5) generating a partial vacuum, a suction head (7) that is fitted with at least one recess (2) having a lateral aperture,
characterized in that
tissue attachment at the appropriate site of the apparatus of the invention is detected by an attachment detection system (12) and is displayed by a display unit (15).

2. Apparatus as claimed in claim 1, characterized in that the attachment detection system (12) comprises at least one detector measuring a pressure or a change in pressure.

3. Apparatus as claimed in claim 1, characterized in that a pressure sensor (15) is used to detect a pressure or a change in pressure.

4. Apparatus as claimed in claim 1, characterized in that the attachment detection system (12) comprises an optical detector to detect tissue/organ attachment.

5. Apparatus as claimed in claim 1, characterized in that an interruptible light beam (13a) and/or an endoscope (13b) and/or a combined optic-electric detector in the form of electrically conducting fiber optics is used as the optical detector.

6. Apparatus as claimed in claim 1, characterized in that the attachment detection system (12) comprises an acoustic and/or electrical detector to detect attachment of a tissue or organ.

7. Apparatus as claimed in claim 1, characterized in that an ultrasonic sensor (14) and/or electrodes are used as acoustic and/or electrical detectors of tissue or organ attachment in the attachment detection system (12), both detectors preferably being designed to identify the tissue/organ and, in the case of electrodes, same in especially preferred manner also being designed to monitor organ functions.

8. Apparatus as claimed in claim 1, characterized in that the display unit (16) comprises means emitting optical, acoustic, olfactory or tactile (for instance vibrational) signals as the display means.

9. Apparatus as claimed in claim 1, characterized in that the signals are adjustable in intensity (for instance loud, low acoustic volume) and/or in their frequency (acoustics, color of light) in proportion to the degree of attachment.

10. Apparatus as claimed in claim 1, characterized in that the recess (2) having the lateral aperture is elongated, whereby sufficient space is assured for the aspirated tissue/organ (10) to allow successful manipulation of said tissue/organ.

11. Apparatus as claimed in claim 1, characterized in that the vacuum duct within the guide device (6) tapers toward the vacuum aperture (11) in the transition zone to the suction head (7), whereby the more pronounced partial vacuum is generated at the vacuum aperture site.

12. Apparatus as claimed in claim 1, characterized in that the recess is 8.5 mm long, 4 mm wide and 3 mm deep.

13. Apparatus as claimed in claim 1, characterized in that the penetrating element illustratively is a needle, a drill bit, tongs or an electromagnetic source of radiation.

14. Apparatus as claimed in claim 1, designed in a manner that the puncture or manipulation is carried out outside the recess (2) or outside the zone of tissue attachment to the suction head (7).

15. Apparatus as claimed in claim 1, characterized in that it comprises at least one flexible or rigid guide device (6) fitted with several lumens to both guide an element (1) designed to puncture or manipulate tissue or organs and to separately guide detectors, said apparatus being fitted with a suction head (7) which can be repeatedly detached from the guide device (6) and a control unit (4) which can be repeatedly detached from said guide device and which drives the penetrating element (1).
